(19) United States
(12) Patent Application Publication
(10) Pub. No.: US 2022/0371958 A1
(43) Pub. Date: Nov. 24, 2022

(54) BIOMATERIAL COMPOSITE, PEPTIDE-BASED ADHESIVES AND METHODS OF USE THEREOF

(71) Applicant: King Abdullah University of Science and Technology, Thuwal (SA)

(72) Inventors: Charlotte A.E. Hauser, Thuwal (SA); Carlos M. Duarte, Thuwal (SA); Sebastian Schmidt-Roach, Thuwal (SA); Manuel Aranda Lastra, Thuwal (SA)

(21) Appl. No.: 17/767,351

(22) PCT Filed: Oct. 7, 2020
(86) PCT No.: PCT/IB2020/059419

§ 371 Date: Apr. 7, 2022

Related U.S. Application Data
(60) Provisional application No. 62/912,032, filed on Oct. 7, 2019.
(60) Provisional application No. 62/954,424, filed on Dec. 28, 2019.
(60) Provisional application No. 62/954,429, filed on Dec. 28, 2019.

(51) Int. Cl. C04B 26/02; B33Y 10/00; B33Y 80/00; C07K 5/103; E02B 3/04

(52) U.S. Cl. CPC C04B 26/026; B33Y 10/00; B33Y 80/00; C07K 5/101; E02B 3/046; C04B 2111/00181

(57) ABSTRACT

Compositions for making native-like marine structures are provided. The compositions include materials, typically a limestone and/or ceramic composite, for 3D printing of marine structures. Environmentally-friendly 3D printing and coating methods are also provided. The methods can be used to print structures/objects composed of a 3D printing material such as the compositions described herein, and/or coat formed structures/objects with an adhesive, such as a peptide-based bioorganic adhesive. Compositions and methods for supporting the attachment and growth of marine organisms such as coral to a substrate are also provided. The compositions contain one or more self-assembling peptides suitable for use as bioorganic adhesives. The peptide compositions are suitable for adhesive applications in the marine environment such as supporting coral growth and restoration. Methods of using the peptide-based adhesive compositions are also provided.

### CROSS-REFERENCE TO RELATED APPLICATION

The present application claims priority to U.S. Application No. 62/912,032, filed Oct. 7, 2019, U.S. Application No. 62/954,429 filed Dec. 28, 2019, and U.S. Application No. 62/954,424, filed Dec. 28, 2019, the disclosures of which are incorporated herein by reference in their entirety.

### FIELD OF THE INVENTION

The field of the invention generally relates to compositions including materials and peptide-based adhesive compositions and methods fabricating native-like marine structures.

### BACKGROUND OF THE INVENTION

Healthy coral reefs are among the most biologically diverse and economically valuable ecosystems on Earth, providing valuable and vital ecosystem services. Coral ecosystems are a source of food for millions; protect coastlines from storms and erosion; provide habitat, spawning and nursery grounds for economically important fish species; provide jobs and income to local economies from fishing, recreation, and tourism; are a source of new medicines, and are hotspots of marine biodiversity.

Unfortunately, coral reefs are at great risk. More than 30% of the world's coral reefs died over the past several decades, and over 90% of surviving reefs are projected to die by 2050. In regions like the Caribbean, over 80% have already died. Stressors include a combination of shifting ocean temperatures, acidification, overfishing, pollution, and poorly managed coastal development.

Extensive efforts are underway for the preservation and/or restoration of coral reefs including, underwater coral farming and reattaching broken coral pieces, rearing of coral fragments in coral nurseries, transplantation of these fragments to degraded reef areas, and subsequent management and monitoring to facilitate restoration.

There is an ongoing need for innovative ways to further support coral restoration, such as construction of artificial reefs that increase the amount of reef structure and habitat available for the corals and other reef organisms to grow on.

It is an object of the invention to provide compositions and methods to support growth and restoration of corals.

It is an object of the invention to provide compositions and methods to enhance the adherence of corals to artificial substrates and allow their growth in underwater conditions.

It is an object of the invention to provide materials for 3D printing of native-like marine structures.

It is yet another object of the invention to provide 3D printing and coating methods to fabricate native-like marine structures.

### SUMMARY OF THE INVENTION

Compositions for making native-like marine structures are provided. The compositions include materials, typically a limestone and/or ceramic composite, for 3D printing of marine structures. In some embodiments, the composition is or contains limestone. For example, the composition can be a limestone-ceramic composite. Preferably, the composition does not contain cement.

The composition can be in the form of a granular material, such as powder, flake, sand, or a combination thereof.

The composition can be used to fabricate various native-like marine structure, such as corals and shells, via 3D printing, and can be used to fabricate marine tiles for applications in underwater use such as coral restoration.

In some embodiments, the composition is held together by and/or coated with a bioorganic adhesive disclosed herein.

Compositions and methods for supporting the attachment and growth of marine organisms such as coral to a substrate are also provided. The compositions contain one or more self-assembling peptides, modified for use as bioorganic adhesives (herein also, bioadhesive).

In particular, disclosed are compositions for adhering one or more aquatic/marine organisms to a substrate. The compositions include an effective amount of a self-assembling amphiphilic peptide with one or more modifications such as L-3,4-dihydroxyphenylalanine (L-dopa), to form a bioadhesive. The L-dopa residues can be incorporated at the N- and/or C-termini of the peptides. The self-assembling peptide preferably conforms to the formula comprising Z—(X)ₐY_{d} B(X′)_{c}Yₙ′—Z′_{b} (Formula I);

wherein Z is an N-terminal protecting group;

X and X′ are, at each occurrence, independently selected from the group consisting of aliphatic amino acids and aliphatic amino acid derivatives, and wherein the overall hydrophobicity decreases from N- to C-terminus;

a is an integer selected from 0 to 10;

c is an integer selected from 0 to 10, preferably 0, 1 or 2;

d is an integer selected from 0 to 10, preferably 0, 1 or 2;

n is preferably 1 or 2;

X or X′ is present;

B can be absent, and if present, is an aromatic amino acid, such as phenylalanine or tryptophan or an aliphatic counterpart of said aromatic amino acid, such as cyclohexylalanine; beta-cyclohexyl-L-alanine, 4-hydroxy-cyclohexylalanine; and 3,4-dihydroxycyclohexylalanine,

Y and Y′, at each occurrence, independently selected from the group consisting of polar amino acids and polar amino acid derivatives; and

Z′ is a C-terminal group; and b is 0 or 1.

Suitable aliphatic amino acids include alanine (Ala, A), homoallylglycine, homopropargylglycine, norleucine, leucine (Leu, L), valine (Val, V) and glycine (Gly, G). Preferably, aliphatic amino acids are selected from alanine (Ala, A), isoleucine (He, I), leucine (Leu, L), valine (Val, V) and glycine (Gly, G).

Exemplary sequences of the self-assembling peptides that fall within the scope of Formula I include, without limitation, IVZK—NH2 (Ile-Val-Cha-Lys-NH2) where Z/Cha is beta-cyclohexyl-L-alanine (SEQ ID NO:1), IIZK (SEQ ID NO:2), IVFK (SEQ ID NO:2), IFVK (SEQ ID NO:4), FIVK (SEQ ID NO:5), FVIK (SEQ ID NO:6), IVFD (SEQ ID NO:7), KIVF (SEQ ID NO:8), KVFI (SEQ ID NO:9) (where B is present) and ILVAGD (SEQ ID NO:10), LIVAGD (SEQ ID NO:11), LIVAAD (SEQ ID NO:12), ILVAGD (SEQ ID NO:13), ILVAGK (SEQ ID NO:14), ALVAG (SEQ ID NO:15), LAVAGD (SEQ ID NO:6), AIVAGD (SEQ ID NO:17), LIVAGE (SEQ ID NO:18), LIVAGS (SEQ ID NO:19), ILVAGS (SEQ ID NO:20), AIVAGS (SEQ ID NO:21), LIVAGT (SEQ ID NO:22) and AIVAGT (SEQ ID NO:23).

Exemplary L-DOPA modified self-assembling peptides which self-assemble into a bioadhesive as described herein include of DopaIIZK (SEQ ID NO: 24), IIZKDopa (SEQ ID NO: 25), IIZDopaK (SEQ ID NO: 26), IIZ(KDopa)₂ (SEQ ID NO: 27), and IIZ(KDopa)₃ (SEQ ID NO: 28).

In some embodiments, the peptide is in the form of a solution, powder or gel (e.g., a hydrogel). The composition of the peptides can contain a mesh or network of fibers of the peptide. In some embodiments, the peptides in the compositions are self-assembled. The composition can be applied to the substrate by any suitable means. In some embodiments, the composition containing the self-assembling peptides is applied to one or more surfaces of the substrate by spraying.

The one or more aquatic/marine organisms can be selected from corals, clams, sponges, or algae. Preferably, the organism is a coral. Suitable substrates upon which the aquatic/marine organisms are deposited, attached and/or adhered via the peptide-based adhesive include ceramics, 3D printed structures, marine structures, limestone, and limestone and/or ceramic composites.

Environmentally-friendly 3D printing and coating methods are also provided. The methods can be used to print structures/objects composed of a 3D printing material such as the compositions described herein, and/or coat formed structures/objects with an adhesive, such as a peptide-based bioorganic adhesive. In some embodiments, the 3D-printing material is combined with a bioorganic adhesive prior to or during the 3D-printing process. In some embodiments, the 3D-printing process involves solid material printing and/or robotic-driven soft material printing.

Also provided are methods for supporting coral growth and restoration using any of the aforementioned peptides and compositions thereof. An exemplary method includes providing a substrate for coral attachment; applying a composition including an effective amount of one or more self-assembling peptides to the surface(s) of the substrate; depositing coral upon the coated surface(s); and placing the substrate in an environment suitable for the growth of the coral. In some embodiments, the substrate is a 3D printed substrate.

### BRIEF DESCRIPTION OF THE DRAWINGS

FIG. 1 is a schematic showing the process including time frame, from a printed coral structure, to the coral structure including a coral fragment

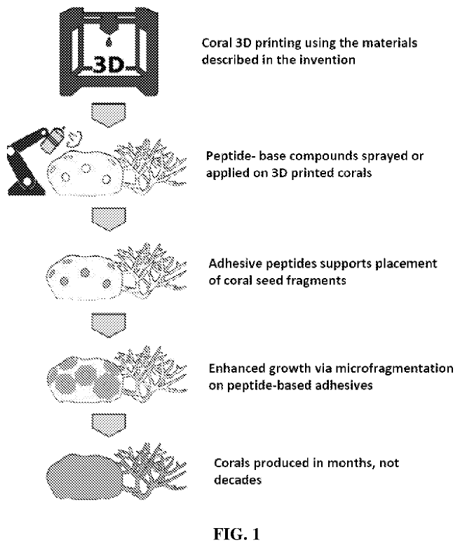

FIG. 2 is a schematic showing the process from a printed coral structure, to the coral structure including a coral fragment.

FIG. 3 shows the scheme in FIG. 2, with the additional step of applying a peptide composition to the coral. The solution which is used for spraying or for regular deposition can be the same, i.e., there is need to do any modifications in order to use the bioadhesives as a spray.

FIG. 4A shows removal of coral fragment from a healthy coral. FIG. 4B shows a coral fragment. FIG. 4C shows an exemplary peptide-based adhesive prepared from DopaIIZK (SEQ ID NO: 24), IIZKDopa (SEQ ID NO: 25), IIZDopaK (SEQ ID NO: 26), IIZ(KDopa)₂ (SEQ ID NO: 27), and IIZ(KDopa)₃ (SEQ ID NO: 28). FIG. 4D shows the effect of gluing coral fragments glued to tiles with peptide-adhesives formed with peptides with (IIZKDopa; SEQ ID NO: 25) or without an L-Dopa moiety. FIG. 4E shows a printed coral structure and a live coral fragment.

FIG. 5 shows a printed coral structure with a live coral fragment.

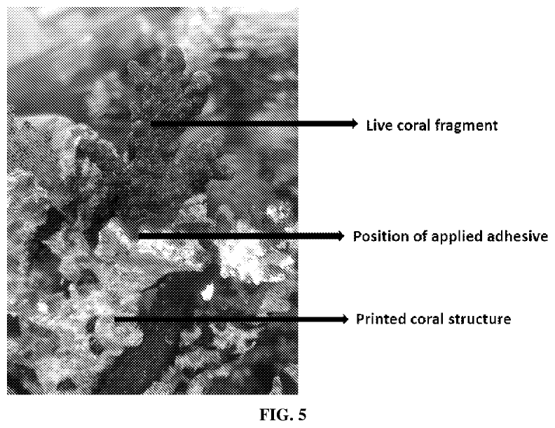

### DETAILED DESCRIPTION OF THE INVENTION

### I. Definitions

As used herein, the terms “self-assemble” and “self-assembly” refer to formation of a discrete, non-random, aggregate structure from component parts; said assembly occurring by induction or spontaneously through random movements of the components (e.g. molecules) due to the inherent chemical or structural properties of those components.

As used herein, the term “variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “aliphatic” means, unless otherwise stated, a straight or branched hydrocarbon chain, which may be saturated or mono- or poly-unsaturated and include heteroatoms. The term “heteroatom” as used herein means an atom of any element other than carbon or hydrogen. An unsaturated aliphatic group contains one or more double and/or triple bonds (alkenyl or alkynyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3 dimethylbutyl. Both the main chain as well as the branches may furthermore contain heteroatoms as for instance N, O, S, Se or Si or carbon atoms may be replaced by these heteroatoms. An aliphatic moiety may be substituted or unsubstituted with one or more functional groups. Substituents may be any functional group, as for example, but not limited to, amino, amido, azido, carbonyl, carboxyl, keto, cyano, isocyano, dithiane, halogen, hydroxyl, nitro, organometal, organoboron, seleno, silyl, silano, sulfonyl, thio, thiocyano, trifluoromethyl sulfonyl, p-toluenesulfonyl, bromobenzenesulfonyl, nitrobenzenesulfonyl, and methanesulfonyl.

The term “hydrophobic” refers to a compound that is soluble in non-polar fluids. The hydrophobic properties of the peptide are due to the presence of non-polar moieties within the same peptide. Besides the hydrophobic peptide sequence part there is a C-terminal —COOH moiety included that can occur in free, unprotected form, or in protected form. Non-polar moieties of a peptide include a hydrocarbon chain that does not carry a functional group.

As used herein, the term “effective amount” means a quantity sufficient to provide a desired effect. For example, an effective amount could be the amount of a disclosed peptide or composition thereof that is sufficient to facilitate adherence of one or more aquatic organisms (e.g., corals) to a substrate. The precise quantity will vary according to a variety of factors such as the substrate, the environmental conditions, the organisms to be adhered, the mode of application, etc.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

### II. Compositions

Solid materials for 3D printing of native-like marine structures including, but not limited to, corals and shells, are provided. The solid materials can include but are not limited to limestone materials, ceramic, or composite materials. Compositions of peptides suitable for use as bioorganic adhesives are also provided.

A. Solid Materials for 3D Printing

Preferably, the 3D printed structures do as little damage to the environment as possible. Thus, currently used eco-unfriendly material for coral reconstruction, such as concrete, polymers, or metals are avoided.

Instead, 3D printing of marine structures is performed using an environmental-friendly solid material such as a limestone composite and/or a ceramic material that integrates natural limestone, which is the native component of the solid sea coral structures.

In some embodiments, the solid compositions for 3D printing of native-like marine structures also contain one or more inorganic components as excipients, such as silica, alumina, and calcium sulfate.

In some embodiments, the solid compositions for 3D printing of native-like marine structures are not cement (e.g., lime-based cement, calcium silicate-based cement, or calcium aluminate-based cement) or do not contain cement as a major component (e.g., less than 50%, 20%, or 10% of the total weight of the solid compositions) if the compositions are composite materials.

In some embodiments, the solid compositions for 3D printing of native-like marine structures are not concrete or do not contain concrete as a major component (e.g., less than 50%, 20%, or 10% of the total weight of the compositions) if the compositions are composite materials.

The solid compositions can be in the form of a granular material, such as powder, flakes, sand, or combinations thereof.

Typically, the printed structure is biocompatible with marine life, and can support, for example, coral growth and a habitat for other marine animals and/or plants.

The solid materials for 3D printing of marine structures can include limestone, or a suitable substitute thereof. Limestone is a carbonate sedimentary rock that is often composed of the skeletal fragments of marine organisms such as coral, foraminifera, and molluscs. Its major materials are the minerals calcite and aragonite, which are different crystal forms of calcium carbonate (CaCO₃). A closely related rock is dolomite, which contains a high percentage of the mineral dolomite, CaMg(CO₃)₂. In some embodiments, dolomite is used in addition or alternative to the limestone in the disclosed composites/compositions.

In some embodiments, the solid materials for 3D printing of marine structures can include other compositions or minerals that contain calcium carbonate, such as calcite, aragonite, vaterite, marble, travertine, chalk, eggshells, and seashells (e.g., oyster shells).

In some embodiments, the solid materials for 3D printing of marine structures can also include lime. Lime is a general term used for various forms of a basic chemical produced from calcium carbonate rocks such as limestone and dolomite. For example, “quicklime” is calcium oxide or calcium-magnesium oxide. “Hydrated lime” (also called “slaked lime”) is produced by mixing the oxide forms with water. “Hydraulic lime” is an impure form of lime that will harden under water. Lime is usually produced by calcining (burning) limestone or dolomite. For example, if limestone is burned at 1010 to 1345° C., the carbon dioxide is driven off and leaves calcium oxide or quicklime

The solid materials for 3D printing of marine structures can include a ceramic material. A ceramic is a solid material including an inorganic compound of metal, non-metal or metalloid atoms primarily held in ionic and covalent bonds. Common examples are earthenware, porcelain, and brick. The crystallinity of ceramic materials ranges from highly oriented to semi-crystalline, vitrified, and often completely amorphous (e.g., glasses). Most often, fired ceramics are either vitrified or semi-vitrified as is the case with earthenware, stoneware, and porcelain. Varying crystallinity and electron composition in the ionic and covalent bonds cause most ceramic materials to be good thermal and electrical insulators (extensively researched in ceramic engineering). With such a large range of possible options for the composition/structure of a ceramic (e.g., nearly all of the elements, nearly all types of bonding, and all levels of crystallinity), the breadth of the subject is vast, and identifiable attributes (e.g., hardness, toughness, electrical conductivity, etc.) are difficult to specify for the group as a whole. General properties such as high melting temperature, high hardness, poor conductivity, high moduli of elasticity, chemical resistance and low ductility are the norm, with known exceptions to each of these rules (e.g., piezoelectric ceramics, glass transition temperature, superconductive ceramics, etc.).

Optionally, the ceramic is or contains a metal oxide. Optionally, the ceramic is a calcium phosphate ceramic. The calcium phosphate ceramic may contain one or more of the following calcium phosphates: monocalcium phosphate, dicalcium phosphate, tricalcium phosphate, octacalcium phosphate, amorphous calcium phosphate, or dicalcium diphosphate (e.g., dicalcium phosphate anhydrous, dicalcium phosphate dihydrate), calcium triphosphate, hydroxyapatite, apatite, carbonated apatite, calcium pyrophosphate, a hydroxyapatite/calcium carbonate mixture, biphasic calcium phosphate, □-tricalcium phosphate, and tetracalcium phosphate. The solid materials for 3D printing of marine structures can be a composite material (also referred to as a composite). A composite is made from two or more constituent materials with different physical or chemical properties that, when combined, produce a material with characteristics different from the individual components. The individual components typically remain separate and distinct within the finished structure, differentiating composites from mixtures and solid solutions. The composite material may be preferred for many reasons, such as, for example, stronger, lighter, or less expensive when compared to one or both of the original materials, or other alternatives thereto.

The disclosed composites typically include limestone, a ceramic material, or a combination thereof. In preferred embodiments, the composites are limestone-ceramic composites. In some embodiments, the composites contain one or more inorganic components as excipients, such as silica, alumina, and calcium sulfate.

In some embodiments, the composites contain limestone in the amount larger than 10%, preferably larger than 20%, more preferably larger than 50%, and most preferably larger than 80%, of the total weight of the composites.

The limestone and/or ceramic-based biomaterial composites can be used for, for example, 3D printing of versatile corals, shells and other marine structures, which in turn are useful for all kind of underwater processes. For example, the printed structures and materials can be used for coral restoration and coral gardening projects as well as underwater architecture projects, i.e., maritecture. In specific examples, the printed structures and materials form part or all of an architectural feature to be placed on underwater structures and/or to be used in coral nursery efforts. Further, marine construction projects include, for example, eco-tourism projects that aim to attract tourists and divers projects that aim to compensate for habitat lost during construction.

In order to cope with the harsh seawater conditions, an adhesive material is needed that tolerates the 3D-printed solid marine structures, and allows biomass such as biological coral tissues to firmly adhere thereto. The adhesive serves one or both of the following two functions: (1) hold together the solid 3D-printing materials, such as those disclosed herein, during 3D printing of native-like marine structures; (2) coat the 3D-printed marine structures, either partially or entirely, so that they can adhere to other native or artificial objects and/or allow marine biomass to be attached.

Preferably, the adhesive can support coral growth and restoration. Preferably, the adhesive is a soft material, such as a liquid or semi-solid. Suitable adhesives include epoxies (such as Epoxo 88, Epoxy 10-3070, BIO-FIX™911, REPAIRITQUIK®, and FASTWELD®), silicone adhesives (such as BIOHESIVE® 225), polyurethane adhesives (such as AQUAHESIVE® 5836), hot-melts or thermoplastic adhesives (such as polyamides, polyolefins, reactive urethane, and ethyl vinyl chloride), acrylics, and bioorganic adhesives (such as protein/peptide-based bioorganic adhesives).

Preferably, the adhesive is a bioorganic adhesive, such as a protein/peptide-based bioorganic adhesive. These bioorganic adhesives can be used for all kind of underwater attachment processes. For example, the bioorganic adhesives can be used for coral restoration and coral gardening projects as well as underwater architecture projects, i.e., maritecture.

B. Soft Materials

In order to cope with the harsh seawater conditions, an adhesive material is needed that tolerates the 3D-printed solid marine structures, and allows biomass such as biological coral tissues to firmly adhere thereto. The adhesive serves one or both of the following two functions: (1) hold together the solid 3D-printing materials, such as those disclosed herein, during 3D printing of native-like marine structures; (2) coat the 3D-printed marine structures, either partially or entirely, so that they can adhere to other native or artificial objects and/or allow marine biomass to be attached.

Preferably, the adhesive can support coral growth and restoration. Preferably, the adhesive is a soft material, such as a liquid or semi-solid. Suitable adhesives include epoxies (such as Epoxo 88, Epoxy 10-3070, BIO-FIX™ 911, REPAIRITQUIK®, and FASTWELD®), silicone adhesives (such as BIOHESIVE® 225), polyurethane adhesives (such as AQUAHESIVE® 5836), hot-melts or thermoplastic adhesives (such as polyamides, polyolefins, reactive urethane, and ethyl vinyl chloride), acrylics, and bioorganic adhesives (such as protein/peptide-based bioorganic adhesives).

Preferably, the adhesive is a bioorganic adhesive, such as a protein/peptide-based bioorganic adhesive. These bioorganic adhesives can be used for all kind of underwater attachment processes. For example, the bioorganic adhesives can be used for coral restoration and coral gardening projects as well as underwater architecture projects, i.e., maritecture.

In some embodiments, the bioorganic adhesives are based on small peptide compounds that are able to self-assemble into nanofibrous networks, such as hydrogels. Accordingly, compositions of peptides suitable for use as bioorganic adhesives are provided. The disclosed adhesives are suitable for adhesive applications in the marine environment. For example, the peptide-based bioorganic adhesives can be used to support coral growth and restoration. The adhesives allow for coral tissue to firmly adhere to various substrates (e.g., ceramic tiles, 3D printed solid structures. The disclosed adhesives are able to withstand the harsh marine environment including mechanical forces and salinity that could otherwise dislodge coral tissue from supportive substrates.

In some embodiments, a biopolymeric gel-like material derived from the disclosed peptides is used as the bioorganic adhesive and is compatible with 3D printed solid coral structures, allows for firm adherence of coral tissue to substrates and other structures, and can cope with the harsh seawater conditions. These adhesives are able to attach corals to surfaces such as ceramics.

i. Peptides

The bioorganic adhesives are based on small peptides which are amphiphlic. Accordingly, the disclosed compositions of bioorganic adhesive contain one or more amphiphlic peptides. The amphiphlic peptides preferably include at least one L-Dopa modification. The peptides preferably include at least one L-Dopa modification and do not include functionalization with biotin or a biotin modification. The disclosed peptide sequences in some preferred embodiments do not include the amino acid Phe or an Fmoc protective group.

In some embodiments, the peptides conform to a peptide motif that enables small peptides (e.g., with 4-8 amino acids, e.g., 3-6 amino acids) to self-assemble to helical fibers in supramolecular structures. In some embodiments, this peptide motif includes a tail of aliphatic nonpolar amino acids (N terminus) c head group of acidic, neutral, or basic nonaromatic polar amino acids (C terminus) (SEQ ID NOS. 10-23 and for example Hauser, et al., Proc. Natl. Acad. Sci. USA, 108(4):1361-6 (2011)). The length of the hydrophobic tail and the polarity of the head group are integral elements that support facile hydrogel formation. In some embodiments, hexamers can form gels more readily than pentamers, tetramers, and trimers. It has been observed that stronger gels are derived from head groups with acidic (D and E), followed by neutral (S and T) and basic (K) polar, nonaromatic amino acids. In some embodiments, the sequence motif starts from N terminus to C terminus in the order of leucine (L) [or isoleucine (I)], followed by isoleucine (or leucine), valine (V), alanine (A), and glycine (G) to guarantee the favorable decrease of nonpolar character toward the polar C terminus. This arrangement of amino acids gives rise to cone-like structures that are prone to assemble noncovalently by molecular recognition in a parallel-antiparallel stacked fashion.

In some embodiments, the peptides from which the bioorganic adhesives are based have the general formula:

Z—(X)ₐY_{d} B(X′)_{c}Yₙ′—Z′_{b} (Formula I);

wherein Z is an N-terminal protecting group;

X and X′ are, at each occurrence, independently selected from the group consisting of aliphatic amino acids and aliphatic amino acid derivatives, and wherein the overall hydrophobicity decreases from N- to C-terminus;

a is an integer selected from 0 to 10;

c is an integer selected from 0 to 10, preferably 0, 1 or 2;

d is an integer selected from 0 to 10, preferably 0, 1 or 2;

n is preferably 1 or 2;

X or X′ is present;

B can be absent, and if present, is an aromatic amino acid, such as phenylalanine or tryptophan or an aliphatic counterpart of said aromatic amino acid, such as cyclohexylalanine; beta-cyclohexyl-L-alanine; 4-hydroxy-cyclohexylalanine; and 3,4-dihydroxycyclohexylalanine,

Y and Y′, at each occurrence, independently selected from the group consisting of polar amino acids and polar amino acid derivatives; and

Z′ is a C-terminal group; and b is 0 or 1.

In some embodiments, X is present and X′ is absent, and vice versa, and/or Y is present and Y is absent, and vice versa.

The peptides typically have short sequences (e.g., 2-10 amino acids). In some embodiments, the peptides contain 2, 3, 4, 5, 6, 7, 8, 9, 10 amino acids. Preferably, the peptides have short sequences of 4-8 amino acids (e.g., 4, 5, 6, 7, 8 amino acids).

The polar amino acid is preferably selected from the group consisting of aspartic acid, asparagine, glutamic acid, glutamine, serine, threonine, methionine, arginine, histidine, lysine, ornithine (Orn), 2,4-diaminobutyric acid (Dab), and 2,3-diaminopropionic acid (Dap).

In some embodiments, aliphatic amino acids and aliphatic amino acid derivatives exhibit an overall decrease in hydrophobicity from the N-terminus to the C-terminus of the peptide (e.g., in order to form nanofibrous hydrogels). The aliphatic amino acids and aliphatic amino acid derivatives can be either D-amino acids or L-amino acids. In some embodiments, the aliphatic amino acids are selected from alanine (Ala, A), homoallylglycine, homopropargylglycine, isoleucine (Ile, I), norleucine, leucine (Leu, L), valine (Val, V) and glycine (Gly, G). In preferred embodiments, the aliphatic amino acids are selected from alanine (Ala, A), isoleucine (Ile, I), leucine (Leu, L), valine (Val, V) and glycine (Gly, G).

The aliphatic amino acids can be arranged in an order of decreasing amino acid size and/or have a sequence which is a non-repetitive sequence. The very first N-terminal amino acid of the aliphatic amino acids can be variable (e.g., it can be G, V or A). This specific first amino acid is not dominant on the requirement of decreasing hydrophobicity from N- to C-terminus. In some embodiments, the first N-terminal amino acid of the aliphatic amino acids is G, V or A. Y is, at each occurrence, independently selected from the group consisting of polar amino acids and polar amino acid derivatives.

In some embodiments, all or a portion of the aliphatic amino acids are arranged in an order of identical amino acid size. Preferably, when the aliphatic amino acids are arranged in order of identical amino acid size, they include a sequence with a length of 2 to 4 amino acids.

Exemplary peptides suitable for use in the bioorganic adhesive compositions are ILVAGD (SEQ ID NO:10), LIVAGD (SEQ ID NO:11), LIVAAD (SEQ ID NO:12), ILVAGD (SEQ ID NO:13), ILVAGK (SEQ ID NO:14), ALVAG (SEQ ID NO:15), LAVAGD (SEQ ID NO:6), AIVAGD (SEQ ID NO:17), LIVAGE (SEQ ID NO:18), LIVAGS (SEQ ID NO:19), ILVAGS (SEQ ID NO:20), AIVAGS (SEQ ID NO:21), LIVAGT (SEQ ID NO:22) and AIVAGT (SEQ ID NO:23) Amphiphilic peptides that self-assemble can be used as a peptide adhesives after they have been modified with L-DOPA, as disclosed herein. Exemplary amphiphilic peptides that self-assemble and can be used as a peptide adhesive after modification with L-DOPA, are disclosed in U.S. Published application 2020/0148720, therein, SEQ ID NOS. 1-128 (incorporated herein by reference), examples reproduced herein, including, but not limited to IVZK (SEQ ID NO:1), IIZK (SEQ ID NO:2), IVFK (SEQ ID NO:2), IFVK (SEQ ID NO:4), FIVK (SEQ ID NO:5), FVIK (SEQ ID NO:6), IVFD (SEQ ID NO:7), KIVF (SEQ ID NO:8), KVFI (SEQ ID NO:9) etc. amphiphilic peptide sequences show true supergelating properties, forming low molecular weight gels (LMWGs) by entrapping a solvent, e.g. water or other aqueous solutions, such as physiological buffers, of over 99% by weight. Interestingly, these amphiphilic peptides have an innate propensity to self-assemble to three dimensional (3D) fibrous networks in form of hydrogels. These gels can also be termed nanogels, because the diameter of the single fibers of the gel's fiber network have nanometer diameters. These peptide compounds are self-driven by non-covalent interactions to form soft solid material.

The compositions of the disclosed peptides can be in any form suitable for use in accordance with any of the disclosed methods or uses. For example, the compositions can be in the form of a dry powder, a wafer, a disk, a tablet, a capsule, a liquid, a gel, a cream, a foam, an ointment, an emulsion, a coating (e.g., on a substrate), or a hydrogel. Preferably, the peptides are formulated as a solution (e.g., an aqueous solution, or a solution in phosphate buffered saline (PBS)), a powder, or gel.

ii. Peptide Modifications

The disclosed peptides may be modified in various ways. In some embodiments, the modification(s) may render the peptides more stable (e.g., resistant to degradation), or confer other desirable characteristic as will be appreciated by one skilled in the art.

Such modifications include, without limitation, chemical modification, N terminus modification, C terminus modification, peptide bond modification, backbone modifications, residue modification, D-amino acids, or non-natural amino acids or others. Any peptide of the disclosure can contain one or more modifications. Specifically disclosed are the variants and/or derivatives of the peptides of SEQ NOs. 1-23 containing one or more modifications described herein (e.g., acetylation, L-Dopa incorporation). Also specifically disclosed are the peptides having a sequence of amino acids selected from IVZK (SEQ ID NO:1), IIZK (SEQ ID NO:2), IVFK (SEQ ID NO:2), IFVK (SEQ ID NO:4), FIVK (SEQ ID NO:5), FVIK (SEQ ID NO:6), IVFD (SEQ ID NO:7), KIVF (SEQ ID NO:8), KVFI (SEQ ID NO:9) (where B is present) and ILVAGD (SEQ ID NO:10), LIVAGD (SEQ ID NO:11), LIVAAD (SEQ ID NO:12), ILVAGD (SEQ ID NO:13), ILVAGK (SEQ ID NO:14), ALVAG (SEQ ID NO:15), LAVAGD (SEQ ID NO:6), AIVAGD (SEQ ID NO:17), LIVAGE (SEQ ID NO:18), LIVAGS (SEQ ID NO:19), ILVAGS (SEQ ID NO:20), AIVAGS (SEQ ID NO:21), LIVAGT (SEQ ID NO:22) and AIVAGT (SEQ ID NO:23), each containing one or more modifications described herein (e.g., acetylation, L-Dopa incorporation).

In a particularly preferred embodiment, the self-assembling L-DOPA containing peptides disclosed herein, including the exemplified amphiphilic peptides, do not include/are not modified with a fluorenylmethyloxycarbonyl (Fmoc) group. The disclosed peptides preferably do not include use biological toxic/undesired moieties such as Fmoc which doesn't exist in biological entities, when applied to coral. Thus, the disclosed peptides sequences preferably do not include the use of biological toxic/undesired and unnatural moieties such as Fmoc, when employed as adhesives on corals.

The peptides of the disclosure may contain naturally occurring α-amino acid residues, non-naturally occurring α-amino acid residues, and combinations thereof. The D-enantiomer (“D-α-amino acid”) of residues may also be used Amino acids useful for inclusion in the disclosed peptides include, but are not limited to, naturally occurring amino acids and artificial amino acids. Incorporation of artificial amino acids such as beta or gamma amino acids and those containing non-natural side chains, and/or other similar monomers such as hydroxyacids are also contemplated, with the effect that the corresponding component is peptide-like in this respect. Non-naturally occurring amino acids are not found or have not been found in nature, but they can by synthesized and incorporated into a peptide chain. Non-natural amino acids are known to those skilled in the art of chemical synthesis and peptide chemistry. Non-limiting examples of suitable non-natural amino acids (each one in L- or D-configuration) are azidoalanine, azidohomoalanine, 2-amino-5-hexynoic acid, norleucine, azidonorleucine, L-α-aminobutyric acid, 3-(1-naphthyl)-alanine, 3-(2-naphthyl)-alanine, p-ethynyl-phenylalanine, m-ethynyl-phenylalanine, p-ethynyl-phenylalanine, p-bromophenylalanine, p-idiophenylalanine, p-azidophenylalanine, and 3-(6-chloroindolyl) alanin.

In some embodiments, peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), CC-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom. These modifications can occur at any of the bonds along the peptide chain and even at several (e.g., 2, 3, 4 or more) at the same time.

In some embodiments, a peptide can have a non-peptide macromolecular group covalently attached to its amino and/or carboxy terminus. Non-limiting examples of such macromolecular groups are proteins, lipid-fatty acid, polyethylene glycol, and carbohydrates. Peptidomimetics may optionally be used to inhibit degradation of the peptides by enzymatic or other degradative processes. The peptidomimetics can be produced by organic synthetic techniques. Non-limiting examples of suitable peptidomimetics include D amino acids of the corresponding L amino acids.

In some embodiments, the peptides of the disclosure contain one or more of the following modifications: glycosylation, amidation, acetylation, acylation, alkylation, alkenylation, alkynylation, phosphorylation, sulphorization, hydroxylation, hydrogenation, cyclization, ADP-ribosylation, anchor formation, covalent attachment of a lipid or lipid derivative, methylation, myristylation, pegylation, prenylation, esterification, biotinylation, coupling of farnesyl or ubiquitination, a linker which allows for conjugation or functionalization of the peptide, or a combination thereof.

Either or both termini of a given linear peptide can be modified. The peptides can be acetylated and/or amidated. In some embodiments for example, the peptides are acetylated at the N-terminus and/or amidated at the C-terminus.

In some embodiments, e.g., Formula I, the peptides have an N-terminal protecting group. The N-terminal protecting group can have the general formula —C(O)—R, where R is selected from H, unsubstituted or substituted alkyls, and unsubstituted or substituted aryls. Preferably, R is selected from methyl, ethyl, propyl, isopropyl, butyl and isobutyl. In some embodiments, the N-terminal protecting group Z is an acetyl group. In some embodiments, the N-terminal protecting group Z is a peptidomimetic molecule, including natural and synthetic amino acid derivatives, where the N-terminus of said peptidomimetic molecule may be modified with a functional group selected from carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

In some embodiments, e.g., Formula I, the peptides have a C-terminal group. The C-terminal group can be a non-amino acid, such as small molecules, functional groups and linkers. Such C-terminal groups can be polar or non-polar moieties used to functionalize the peptide.

In some embodiments, the C-terminal group is selected from functional groups, such as polar or non-polar functional groups, such as (but not limited to) —COOH, —COOR, —COR, —CONHR or —CONRR′ with R and R′ being selected from H, unsubstituted or substituted alkyls, and unsubstituted or substituted aryls, —NH₂, —OH, —SH, —CHO, maleimide, imidoester, carbodiimide ester, isocyanate; small molecules, such as (but not limited to) sugars, alcohols, hydroxy acids, amino acids, vitamins, biotin; linkers terminating in a polar functional group, such as (but not limited to) ethylenediamine, PEG, carbodiimide ester, imidoester; and linkers coupled to small molecules or vitamins, such as biotin, sugars, hydroxy acids.

In some embodiments, the C-terminal group is a peptidomimetic molecule, including natural and synthetic amino acid derivatives, where the C-terminus of said peptidomimetic molecule may be modified with a functional group selected from carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

### L-Dopa Modifications

In preferred embodiments, the peptides include, or are modified with, amino acid L-3,4-dihydroxyphenylalanine (L-dopa). The peptides can include one or more (e.g., 1, 2, 3, 4, 5, or more) L-dopa residues. The peptides can be modified with L-Dopa at any desirable position (e.g., at the termini or internally). For example, the N-terminus and/or the C-terminus of the disclosed peptides can be modified with L-Dopa. In preferred embodiments, the C-terminus of the peptides is modified with L-Dopa. None limiting examples of L-DOPA modified self-assembling peptides which self-assemble into a bioadhesive are DopaIIZK (SEQ ID NO: 24), IIZKDopa (SEQ ID NO: 25), IIZDopaK (SEQ ID NO: 26), IIZ(KDopa)₂ (SEQ ID NO: 27), and IIZ(KDopa)₃ (SEQ ID NO: 28); however, all of the self-assembling peptides which fall within the scope of Formula I, including specific peptide sequences disclosed herein as well as the sequences disclosed in U.S. Published application 2020/0148720, as SEQ ID NOS. 1-128 (incorporated herein by reference), can be similarly modified in terms of the position and number modifications, as demonstrated herein for IIZK.

Marine mussels have mastered the ability to anchor to foreign surfaces in seawater through the use of adhesive proteins. These mussel foot proteins (Mfps) are known to cure rapidly to form adhesive plaques with high interfacial binding strength, durability, and toughness. 3,4-Dihydroxyphenylalanine (Dopa), which is modified from tyrosine through post-transitional hydroxylation, is one of the main constituents in Mfps. L-dopa is seemingly responsible for the strong attachment of mussels, i.e. their adhesion behavior in seawater, being able to stay adherent to underwater surfaces despite seawater turbulences and wave movements. The catechol side chain of Dopa has the ability to form various types of chemical interactions and crosslinking, which imparts Mfps with the ability to solidify in situ and bind tightly to various types of surface substrates. See Kord Forooshani P. and Lee BP., J. Polym. Sci. A Polym. Chem., 55(1):9-33 (2017) for a review of recent approaches in designing bioadhesive materials inspired by mussel adhesive protein.

Mussel adhesives proteins enable marine mussels to attach strongly to various surfaces in their turbulent, wet and saline habitats. These proteins are secreted in a liquid form, which then solidify to form a byssal thread and an adhesive plaque complex. The average force needed to dislodge a California mussel, Mytilus califomianus, is estimated to be 250-300 N/mussel, indicating a remarkable surface anchoring capacity. One of the unique features of Mfps is the abundance of the catecholic amino acid, Dopa, in their protein sequences. The presence of catechol is believed to fulfill the dual role of interfacial binding and the solidification of the adhesive proteins. Catechol is capable of diverse chemistries, which enables it to bind to both organic and inorganic surfaces through the formation of reversible non-covalent or irreversible covalent interactions. The dihydroxy functionality of catechol enables it to form strong hydrogen bonds which promotes its absorption to mucosal tissues and hydroxyapatite surfaces. The benzene ring of catechol is capable of interacting with other aromatic rings through π-π electron interaction which improves the cohesive properties of catechol-containing polymers and enables them to attach to surfaces rich in aromatic compounds (e.g., polystyrene) and gold substrates. The aromatic ring also forms cation-π interaction with positively charged ions, which is one of the strongest non-covalent interactions in water. Cation-π interaction enhances absorption of catechol to charged surfaces and contributes to the cohesive properties of materials rich in both aromatic and cationic functional groups. Since catechols are easily oxidized to its poorly adhesive quinone form in an oxygen rich and basic environment, cation-π interaction complements the underwater adhesive properties of catechol. Kord Forooshani P. and Lee BP., J. Polym. Sci. A Polym. Chem., 55(1):9-33 (2017).

Accordingly, modification of the disclosed peptides with Dopa (e.g., L-dopa) is contemplated for conferring adhesive properties, similar to that of Mfps, to the disclosed peptides and compositions thereof such as hydrogels. Therefore, disclosed are self-assembling peptides containing one or more L-dopa residues.

iii. Self-Assembly

The bioorganic adhesives are based on small peptides that are able to self-assemble into nanofibrous networks. The disclosed peptides assemble into fibers that form mesh-like structures. The fibers can vary in diameter/thickness and/or length. Without being bound by theory, hydrophobic interaction between non-polar portions of peptides are contemplated to assist such self-assembly process. In some embodiments, the peptides undergo a conformational change during self-assembly, e.g., a conformational change from a random coil conformation to a helical intermediate structure (such as a-helical fibrils) to a final beta turn or cross beta conformation, such as fibrils which further aggregate and/or condense into nanofibers (which make up a network). The conformational change can be dependent on the peptide concentration, ionic environment, pH and temperature.

The disclosed peptides can have a characteristic motif that facilitates self-assembly in aqueous conditions, forming porous, nanofibrous scaffolds. Typically, the characteristic motif that drives self-assembly consists of a N-terminus “tail” of 2 to 7 natural aliphatic amino acids, arranged in decreasing hydrophobicity towards the C-terminus. The C-terminus can be functionalized, such as with a functional group (e.g. carboxylic acid, amine, ester, alcohol, aldehyde, ketone, maleimide), small molecules (e.g. sugars, alcohols, vitamins, hydroxyl-acids, amino acids) or short polar linkers. Self-assembly in aqueous conditions occurs when the amino acids pair and subsequently stack into a-helical fibrils. Hydrogels are obtained when further aggregation of the fibrils into 3D networks of nanofibers entrap water.

The time required for effective assembly and/or hydrogel formation can vary depending on the peptide, e.g., about 1 min, about 2 min, about 3 min, about 4 min, about 5 min, about 10 min, about 15 min, about 20 min, about 25 min, about 30 min, about 1 hour, about 2 hours, about 1 day, or about 2 days.

In some embodiments, the peptides exhibit stimuli-responsive gelation such as gelation upon exposure to certain pH, salt concentration and/or temperature. In some embodiments, the peptides form a hydrogel. The hydrogel is formed by self-assembly of the peptide.

In some embodiments, the dissolved peptide is warmed or heated, e.g., to a temperature in the range from 20° C. to 90° C., preferably from about 30° C. to 70° C., more preferably from about 37° C. to 70° C.

In some embodiments, a hydrogel is formed by dissolving at least one peptide in an aqueous solution, such as water, or in a polar solvent, such as ethanol. In some embodiments, the peptide is dissolved at a concentration from 0.01 μg/ml to 100 mg/ml, from 1 mg/ml to 50 mg/ml, or from about 1 mg/ml to about 20 mg/ml.

The peptides can be present in various concentrations. In some embodiments, the peptide in a hydrogel is present at a concentration in the range of from 0.1% to 30% (w/w), preferably 0.1% to 20% (w/w), more preferably 0.1% to 10% (w/w), more preferably 0.1% to 5% (w/w), even more preferably 0.1% to 3% (w/w), with respect to the total weight of said hydrogel.

In some embodiments, the peptide includes basic amino acid(s), such as lysine or lysine-mimetic molecules, preferably amidated basic amino acid(s), as the polar head group and gelation is carried out in the presence of salt at physiological conditions (such as PBS or 0.9% saline and PBS) and/or at a pH above physiological pH, preferably pH 7 to 10 (such as by adding NaOH). In some embodiments, the peptide includes acidic amino acid(s), as the polar head group and gelation is carried out at a pH below physiological pH 7, preferably pH 2 to 6.

A hydrogel can be formed by dissolving the peptide(s) in aqueous solution. Agitation, including mixing such as stirring, and/or sonication may be employed to facilitate dissolving the peptide(s). In some embodiments the aqueous solution of the peptide is exposed to a temperature below ambient temperature, such as a temperature selected from about 2° C. to about 15° C. In some embodiments the aqueous solution of the peptide is exposed to an elevated temperature, i.e. a temperature above ambient temperature. Typically the aqueous solution is allowed to attain the temperature to which it is exposed. The aqueous solution may for example be exposed to a temperature from about 25° C. to about 85° C. or higher, such as from about 25° C. to about 75° C., from about 25° C. to about 70° C., from about 30° C. to about 70° C., from about 35° C. to about 70° C., from about 25° C. to about 60° C., from about 30° C. to about 60° C., from about 25° C. to about 50° C., from about 30° C. to about 50° C. or from about 40° C. to about 65° C., such as e.g. a temperature of about 40° C., about 45° C., about 50° C., about 55° C., about 60° C. or about 65° C. The aqueous solution of the peptide may be maintained at this temperature for a period of about 5 min to about 10 hours or more, such as about 10 min to about 6 hours, about 10 min to about 4 hours, about 10 min to about 2.5 hours, about 5 min to about 2.5 hours, about 10 min to about 1.5 hours or about 10 min to about 1 hour, such as about 15 min, about 20 min, about 25 min, about 30 min, about 35 min or about 40 min

Depending on the amino acids that are included in peptides contained in a hydrogel, a respective hydrogel may be biodegradable. A biodegradable hydrogel gradually disintegrates or is absorbed in vivo over a period of time, e.g., within months or years. Disintegration may for instance occur via hydrolysis, may be catalyzed by an enzyme, and/or may be assisted by conditions to which the hydrogel is exposed.

### III. Methods of Making and Using

The disclosed methods include environmentally-friendly 3D printing and coating methods, as well as methods of making self-assembling peptide adhesives. The methods can be used to fabricate native-like corals, shells, and other marine structures, which are biocompatible with native coral and other marine structures.

A. Methods of Printing and Coating

In order to print solid marine structures, the solid printing material is combined with a soft printing material (“bioink”) such as an adhesive as disclosed herein. The two types of materials can be combined before loading to the 3D printer. Alternatively, the two types of materials can be combined in situ during the 3D printing process.

i. 3D Printing

The 3D printing process can be performed using an extrusion-type 3D printer. For example, a robotic arm 3D bioprinter setup is used in combination with microfluidic pumps, described for example in U.S. Published Application No. 2020/0199514, incorporated herein by reference. A two-inlet nozzle is used for extrusion. In some embodiments, one inlet controls extrusion of the solid 3D-printing material, and the other inlet controls extrusion of the soft 3D-printing material.

The 3D-printing methods can be used to fabricate marine structures using materials for 3D printing as described herein, such as limestone and/or ceramic composites, for example, limestone-ceramic composites.

In some embodiments, the soft printing material or bioink is a peptide-based bioorganic adhesive, optionally in the form of a hydrogel or a diluted solution.

An exemplary 3D printer and printing methods are described in U.S. Published Application No. 2020/0199514, incorporated herein by reference. The robotic printing system disclosed in 2020/0199514, can be used for printing on curved surfaces and also allows for use of the printer with spray functionality. This means that the robotic arm can be used as a provider of adhesive material on the surface of the corals (coating).

Reverse engineering of harvested corals is a potential way of producing artificial corals of similar geometries to facilitate coral restoration and gardening. This is achieved by 3D scanning live coral specimens retrieved from sea dives to obtain a CAD model of the complete coral 3D construction with its complex geometries. Further editing can be done to smoothen out any ruptures in the CAD model before printing. The CAD model is then 3D printed with a biocompatible material (disclosed herein) which can be easily installed in the reef without negatively impacting marine life. Exemplary components for the biocompatible material include Calcium and ceramic-based materials. The 3D printer system consists of two robotics arms—one functioning as a 3D printer and the other functioning as a spray. The CAD model is 3D printed using the robotic arm, after which the second arm sprays the peptide bioadhesive to coat the printed corals and enables the adherence of coral microfragments. The effective design of the spraying mechanism was the twin-external mix-spray nozzle. The nozzle has three inlets, one for the peptide mixed with a solution such as PBS, and the other two with tiny pipes of compressed air coming through. The swirling of the air with the gel happens in the outer orifice, to create enough shear for the formation and dispersion of tiny droplets

### ii. Coating

Naturally-occurring or non-naturally-occurring 3D-printed marine structures can be coated with an adhesive such as the bioadhesives disclosed herein using spray devices such as sprays or robotic-driven spray nozzle. In some embodiments, the non-naturally-occurring 3D-printed marine structures are produced via 3D printing, as described herein. Preferably, the adhesive is a peptide as disclosed herein. The disclosed adhesive peptides can be sprayed directly onto different surfaces using a combination of air spray nozzles. FIG. 13A of 2020/0199514 shows the schematic of the system in which two airbrush nozzles were used to spray at an angle so that both the solutions (peptide and phosphate buffer saline) meet on a certain point to form peptide hydrogel. FIG. 13B of 2020/0199514 shows examples of such air spray nozzles. This environmental-friendly 3D printing and coating methods can be used for the fabrication of native-like coral structures and is useful for all kinds of underwater processes. For example, the methods can be used for making structures that can be used for coral restoration and coral gardening projects as well as underwater architecture projects, i.e., maritechture. In specific examples, the printed and/or coated structures form part or all of an architectural feature to be placed on underwater structures and/or to be used in coral nursery efforts. Further, marine construction projects include, for example, eco-tourism projects that want to attract tourists and diver's projects that aim to compensate for habitat lost during construction.

iii. Other Objects for 3D Printing or Coating

The methods can be also used to fabricate parts or building blocks of a marine tank or marine garden. The methods can be also used to coat marine structures, or parts or building blocks of a marine tank or marine garden, in order to promote adherence of biomass.

In some embodiments, the methods can be used to fabricate or coat marine tiles, which are building blocks for different marine-related structures or architectures. The tiles can be in the composition of a mosaic, which can evoke different patterns.

B. Methods of Making Peptides for Use as Bioadhesives

Self-assembling peptides and compositions of the peptides can be prepared using any techniques known in the art. Synthesis of the peptides is easy and cost-effective. The short sequence (e.g., 4-8 amino acids) of the disclosed peptides implies a lower cost and ease of synthesis and purification compared to other self-assembling peptide technologies.

Peptides are typically synthesized using standard procedures, so any technique in the art suitable to prepare synthetic peptides can be used. The peptides can also be produced by recombinant means (e.g., in bacteria, yeast, fungi, insect, vertebrate or mammalian cells) by methods well known to those skilled in the art.

A peptide, including a polypeptide may be synthesized using an automated polypeptide synthesizer. The peptides can be synthesized using techniques well-known to those skilled in the art, e.g., by standard solid-phase peptide synthesis. Such methods include bench scale solid phase synthesis and automated peptide synthesis in any one of the many commercially available peptide synthesizers. Solid phase synthesis is commonly used and various commercial synthesizers are available, for example automated synthesizers by Applied Biosystems Inc., Foster City, Calif.; Beckman; MultiSyntech, Bochum, Germany etc. Solution phase synthetic methods may also be used, although it can be less convenient. Functional groups for conjugating the peptide of the disclosure to small molecules, label moieties, peptides, or proteins may be introduced into the molecule during chemical synthesis. In addition, small molecules and label moieties/reporter units may be attached during the synthetic process. Preferably, introduction of the functional groups and conjugation to other molecules minimally affects the structure and function of the subject peptide. The peptides can be produced by stepwise synthesis or by synthesis of a series of fragments that can be coupled by similar well known techniques.

Synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, peptides having a C-terminal moiety that may be selected from the group consisting of —H, —OH, —COOH, —CONH₂, and —NH₂ are contemplated for use.

In some embodiments, the peptide is modified. Exemplary modifications include esterification, glycosylation, acylation such as acetylation or linking myristic acid, amidation, phosphorylation, biotinylation, PEGylation, coupling of farnesyl and similar modifications which are well known in the art. Modifications can be effected at the N-terminus, the C-terminus or at any amino acid in between (e.g. farnesyl coupling to a Cys side chain). It is believed that modifications, such as amidation, enhance the stability of the peptide to peptidases. Methods for acylating, and specifically for acetylating the free amino group at the N-terminus are well known in the art. For the C-terminus, the carboxyl group may be modified by esterification with alcohols or amidated to form-CONhb or CONHR. Methods of esterification and amidation are done using well known techniques.

In some embodiments, the peptides are synthesized using standard Fmoc chemistry. Standard Fmoc (9-florenylmethoxycarbonyl) derivatives include Fmoc-Asp(OtBu)-OH, Fmoc-Arg(Pbf)-OH, and Fmoc-Ala-OH. Couplings are mediated with DIC (diisopropylcarbodiimide)/6-Cl-HOBT (6-chloro-1-hydroxybenzotriazole). In some embodiments, the last four residues of the peptide require one or more recoupling procedures. In particular, the final Fmoc-Arg(Pbf)-OH coupling can require recoupling. For example, a second or third recoupling can be carried out to complete the peptide using stronger activation chemistry such as DIC/HOAT (1-hydroxy-7-azabenzotriazole) or HATU (1-[bis(dimethylamino)methylene]-1H-1,2,3-triazolo[4,5-b]pyridinium 3-oxid hexafluorophosphate)/NMM (N-methylmorpholine.

Acidolytic cleavage of the peptide can be carried out with the use of carbocation scavengers (thioanisole, anisole and H₂O). Optimization can be achieved by varying the ratio of the components of the cleavage mixture. An exemplary cleavage mixture ratio is 90:2.5:2.5:5 (TFA-thioanisole-anisole-H₂O). The reaction can be carried out for 4 hours at room temperature.

In some embodiments, the removal of residual impurities is carried out by wash steps. For example, trifluoroacetic acid (TFA) and organic impurities can be eliminated by precipitation and repeated washes with cold diethyl ether and methyl t-butyl ether (MTBE).

Peptides produced using the disclosed methods can be purified using high pressure liquid chromatography (HPLC). In some embodiments, the peptides can be purified >95% via HPLC Amino acid and peptide content analysis can be subsequently performed. Suitable solvents for dissolving the peptides include neat TFA. In some embodiments, 8 mL TFA/g peptide is sufficient to fully dissolve peptides following precipitation. TFA can be diluted into H₂O. Typically, the peptides remain soluble at TFA concentrations of 0.5% to 8% and can be loaded onto reverse phase (RP)-HPLC columns for salt exchange. Exemplary salt exchange methods use 3-4 column volumes of acidic buffer to wash away the TFA counter ion due to its stronger acidity coefficient. Buffers suitable for use in washing away the TFA counter ion include 0.1% HCl in H₂O.

Following removal of TFA, peptides can be eluted with a step gradient. Exemplary elution buffers include 30% acetonitrile (MeCN) vs. 0.1% HCl in H₂O. For acetate exchange, peptides can be loaded from the same diluted TFA solution, washed with 3-4 column volumes of 1% acetic acid (AcOH) in H₂O, followed by 2 column volumes of 0.1 M NH₄OAc in H₂O, pH 4.4. In some embodiments, the column is washed again with 3-4 column volumes of 1% AcOH in H₂O.

Peptides can be eluted from the columns using a step gradient of 30% MeCN vs. 1% AcOH in H₂O. In some embodiments, the elution of peptides can be enhanced by acetate exchange. Exemplary buffers for acetate exchange include 0.1 M NH₄OAc in H₂O, pH 4.4.

Analytical HPLC can be carried out to assess the purity and homogeneity of peptides. An exemplary HPLC column for use in analytical HPLC is a PHENOMENEX® JUPITER® column. In some embodiments, analytical HPLC is carried out using a column and buffer that are heated to a temperature greater than 25° C., for example 25-75° C. In a particular embodiment, analytical HPLC is carried out at temperatures of about 65° C. A step gradient can be used to separate the peptide composition. In some embodiments, the gradient is from 1%-40% MeCN vs 0.05% TFA in H₂O. The change in gradient can be achieved over 20 minutes using a flow rate of 1 ml/min Peptides can be detected using UV detection at 215 nm.

Where the peptides or compositions thereof are required to be sterilized or otherwise processed for the removal of undesirable contaminants and/or micro-organisms, filtration can be used. Filtration can be achieved using any system or procedures known in the art. In some embodiments, filtration removes contaminants or prevents the growth or presence of microorganisms. Exemplary microorganisms and contaminants that can be removed include bacteria, cells, protozoa, viruses, fungi, and combinations thereof. In some embodiments, the step of filtration is carried out to remove aggregated or oligomerized peptides. For example, solutions of the peptides can be filtered to remove assembled peptide structures or oligomers on the basis of size.

In some embodiments of synthesizing the peptides, to functionalize the C-terminus, L-dopa can be incorporated during solid phase peptide synthesis by first reacting the Fmoc protected precursor to the Wang or Rink-amide resin. The final peptide product can be purified using HPLC/MS and then lyophilized. As previously noted, the final L-DOPA-containing peptide sequence used in the disclosed methods of adhering/growing coral in one preferred embodiment does not include Fmoc. Accordingly, if Fmoc is used in the peptide synthesis process, must be removed using methods known in the art, such as doing treatments with nucleophiles, for example, treatment with piperidine.

C. Methods of Using Peptide Adhesive Compositions

The disclosed L-DOPA-modified peptides (preferably self-assemble in the presence of a salt containing solution) to form a bioadhesive. Also, the molar concentration of the adhesive material does matter. The amount of adhesive glue applied to a surface will depend on the size of the coral micro fragment to be glued, and accordingly, it can range from about 100 μl and up to 1 ml, for bigger pieces. Preferred L-DOPA modified self-assembling peptides which self-assemble into a bioadhesive as described herein include of DopaIIZK (SEQ ID NO: 24), IIZKDopa (SEQ ID NO: 25), IIZDopaK (SEQ ID NO: 26), IIZ(KDopa)₂ (SEQ ID NO: 27), and IIZ(KDopa)₃ (SEQ ID NO: 28).

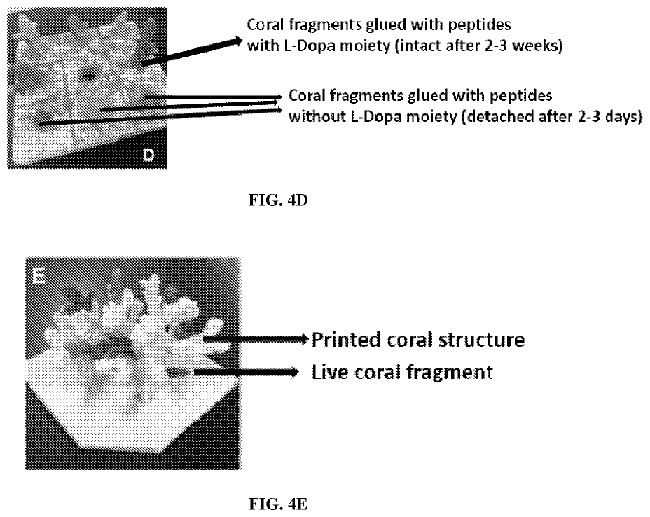

Also provided are uses for the disclosed peptides and compositions thereof, as (a) bioadhesive glue, for adhering coral onto a desired surface and (b) as a coral growth enhancing composition. The peptides, modified with L-Dopa are especially suitable for use as adhesives. The peptide-based adhesives are suitable for adhesive applications in an aqueous (e.g., marine) environment. For example, the peptide-based adhesives can be used to adhere corals. The peptide-based adhesives are able to withstand the harsh marine environment, including mechanical forces and salinity that could otherwise dislodge corals from supportive substrates. This application can support coral growth and restoration efforts. The disclosed peptide adhesives enhance the growth of bioorganic materials (such as soft coral materials, proteins, cell and tissues etc.) when applied onto a desired surface. FIG. 4D compares the growth of coral attached to a surface with a bioadhesive glue that falls within the scope of the invention, with coral attached to a surface with self-assembling peptide, not modified with L-DOPA. When self-assembling peptide without L-Dopa were used, the coral microfragments were detached after few days. On the contrary, fragments that were glued with L-Dopa peptides (bioadhesive) remained intact after weeks.

The bioadhesives allow for coral tissue to firmly adhere to various substrates. Exemplary substrates include, without limitation, ceramics, 3D printed solid structures, marine structures, limestone, limestone and/or ceramic composites (e.g., limestone-ceramic composites), as well as naturally-occurring, and other non-naturally-occurring marine structures. A ceramic is a solid material including an inorganic compound of metal, non-metal or metalloid atoms primarily held in ionic and covalent bonds. Common examples are earthenware, porcelain, and brick. A composite is made from two or more constituent materials with different physical or chemical properties that, when combined, produce a material with characteristics different from the individual components.

The peptides or compositions thereof, e.g., in the form of a solution, powder, or gel, can be used to coat a structure or other substrate, upon which coral tissue is to be deposited/fixed. For example, a solution of one or more peptides can be sprayed over the surface of a substrate. Alternatively, the substrate can be immersed in a solution of the peptide(s). Subsequently, coral can be deposited on the coated surface and allowed to adhere. The substrate can be placed in the appropriate environment to promote and/or sustain growth of the coral.

In some embodiments, a gel or gel-like material derived from the disclosed L-DOPA modified peptides is used as the bioorganic adhesive. The gel or gel-like material typically is compatible with 3D printed solid coral structures, allows for firm adherence of coral tissue to substrates and other structures, and can cope with the harsh seawater conditions. These adhesives are able to attach corals to surfaces such as ceramics.

The disclosed peptide-based adhesives can be used for a variety of underwater attachment processes. For example, the peptide-based adhesives can be used for coral restoration and coral gardening projects as well as underwater architecture projects, i.e. Maritecture. In specific examples, the adhesives form part of an architectural feature to be placed on underwater structures and/or to be used in coral nursery efforts.

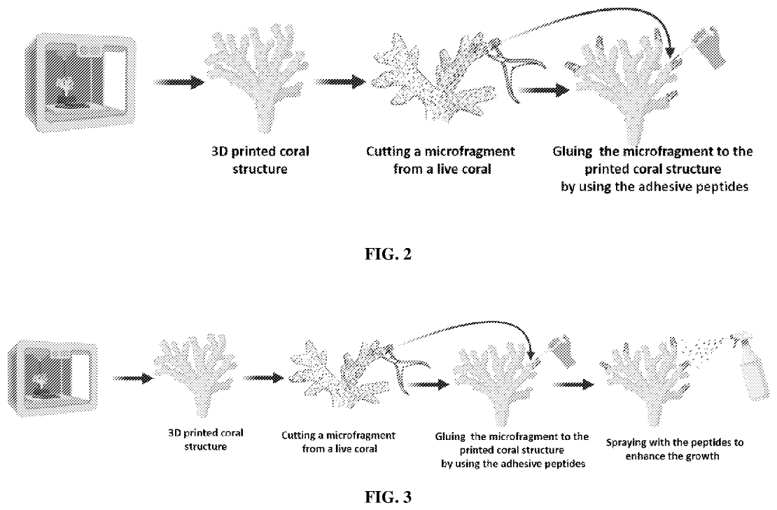

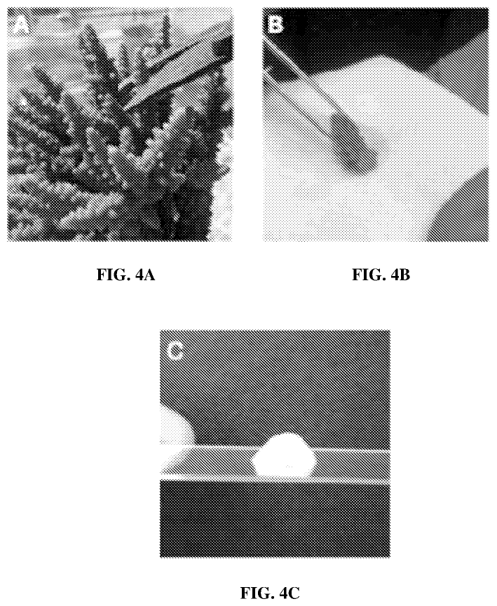

In an exemplary process, a coral structure is 3 D printed, and onto this 3D printed structure is glued a coral microfragment harvested from a live coral (FIG. 2) using the disclosed bioadhesive compositions. As shown in FIG. 3, the disclosed self-assembling peptides can also be applied to the coral microfragment, preferably in the form of a solution, to enhance coral growth.

1. A composition for 3D printing of marine structures comprising limestone, ceramic, or a combination thereof.

2. The composition of claim 1, wherein the composition comprises limestone at an amount that is greater than 80% of the total weight of the composition.

3. The composition of claim 1, wherein: (a) the composition is a composite material; (b) the composition is a limestone-ceramic composite; (c) the composition does not contain cement; and/or (d) the composition is in the form of a granular material.

4. (canceled)

5. (canceled)

6. (canceled)

7. The composition of claim 3, wherein the composition is in the form of powder, flake, sand, or a combination thereof.

8. A 3D-printed artificial marine structure, wherein the marine structure is formed of the composition of claim 1.

9. The 3D-printed artificial marine structure of claim 8, wherein: (a) the marine structure is coral or shell and/or (b) the marine structure is held together by and/or coated with a bioorganic adhesive.

10. (canceled)

11. The 3D-printed artificial marine structure of claim 9, wherein the bioorganic adhesive supports coral growth and restoration and/or the bioorganic adhesive is attached to the surface of the marine structure and/or wherein the bioorganic adhesive comprises one or more peptides containing 10 or less amino acid residues; and/or wherein the bioorganic adhesive is attached to the surface of the marine structure.

12. (canceled)

13. The 3D-printed artificial marine structure of claim 11, wherein: (a) the one or more peptides are self-assembling peptides and optionally, the one or more peptides can self-assemble into a nanofibrous network; (b) the one or more peptides comprise four to eight amino acid residues; and/or (c) the one or more peptides comprise a L-3,4-dihydroxyphenylalanine residue.

14. (canceled)

15. (canceled)

16. (canceled)

17. (canceled)

18. A method for fabricating a marine structure, comprising:
(i) 3D-printing the marine structure using the composition of claim 1, wherein the composition is combined with a first bioorganic adhesive prior to or during step (i), optionally, wherein step (i) comprises solid material printing and/or robotic-driven soft material printing; and/or the marine structure is coral.

19. (canceled)

20. The method of claim 18, further comprising:
(ii) coating the marine structure from step (i) with a second bioorganic adhesive, wherein the second bioorganic adhesive is the same as or different from the first bioorganic adhesive.

21. The method of claim 18, wherein the first bioorganic adhesive, the second bioorganic adhesive, or both comprise one or more peptides containing 10 or less amino acid residues and/or wherein the second bioorganic adhesive is coated on the marine structure from step (i) via spraying.

22. (canceled)

23. (canceled)

24. (canceled)

25. A composition for adhering one or more aquatic organisms to a substrate comprising an effective amount of a self-assembling peptide, comprising one or more L-3,4-dihydroxyphenylalanine (L-dopa) residues

26. The composition of claim 25, wherein the self-assembling peptide conforms to the formula comprising:
Z—(X)ₐY_{d}B(X′)_{c}Yₙ′—Z′_{b} (Formula I);
wherein Z is an N-terminal protecting group or is absent;
X and X′ are, at each occurrence, independently selected from the group consisting of aliphatic amino acids and aliphatic amino acid derivatives, and wherein the overall hydrophobicity decreases from N- to C-terminus;
a is an integer selected from 0 to 10;
c is an integer selected from 0 to 10, preferably 0, 1 or 2;
d is an integer selected from 0 to 10, preferably 0, 1 or 2;
n is preferably 1 or 2;
X or X′ is present;
B is present or absent, and if present, is an aromatic amino acid, such as phenylalanine or tryptophan or an aliphatic counterpart of said aromatic amino acid, such as cyclohexylalanine;
beta-cyclohexyl-L-alanine, 4-hydroxy-cyclohexylalanine; and 3,4-dihydroxycyclohexylalanine, Y and Y′, at each occurrence, independently selected from the group consisting of polar amino acids and polar amino acid derivatives; and
Z′ is a C-terminal group; and b is 0 or 1.

27. The composition of claim 26, wherein: (a) the aliphatic amino acids are selected from the group consisting of alanine (Ala, A), homoallylglycine, homopropargylglycine, isoleucine (Ile, l), norleucine, leucine (Leu, L), valine (Val, V) and glycine (Gly, G), preferably from the group consisting of alanine (Ala, A), leucine (Leu, L), valine (Val, V) and glycine (Gly, G); (b) the polar amino acid is preferably selected from the group consisting of aspartic acid, asparagine, glutamic acid, glutamine, serine, threonine, methionine, arginine, histidine, lysine, ornithine (Orn), 2,4-diaminobutyric acid (Dab), and 2,3-diaminopropionic acid (Dap).

28. (canceled)

29. The composition of claim 25, wherein the peptide is selected from the group consisting of ILVAGD (SEQ ID NO:10), LIVAGD (SEQ ID NO:11), LIVAAD (SEQ ID NO:12), ILVAGD (SEQ ID NO:13), ILVAGK (SEQ ID NO:14), ALVAG (SEQ ID NO:15), LAVAGD (SEQ ID NO:6), AIVAGD (SEQ ID NO:17), LIVAGE (SEQ ID NO:18), LIVAGS (SEQ ID NO:19), ILVAGS (SEQ ID NO:20), AIVAGS (SEQ ID NO:21), LIVAGT (SEQ ID NO:22) and AIVAGT (SEQ ID NO:23).

30. The composition of claim 25, wherein the self-assembling peptide is IVZK (SEQ ID NO:1), IIZK (SEQ ID NO:2), IVFK (SEQ ID NO:2), IFVK (SEQ ID NO:4), FIVK (SEQ ID NO:5), FVIK (SEQ ID NO:6), IVFD (SEQ ID NO:7), KIVF (SEQ ID NO:8), KVFI (SEQ ID NO:9).

31. The composition of claim 25, wherein: (a) the one or more L-dopa residues are incorporated at the N- and/or C-termini of the peptide and optionally comprises a sequence selected from the group consisting of DopaIIZK (SEQ ID NO: 24), IIZKDopa (SEQ ID NO: 25), IIZDopaK (SEQ ID NO: 26), IIZ(KDopa)₂ (SEQ ID NO: 27), and IIZ(KDopa)₃ (SEQ ID NO: 28); (b) the peptide is in the form of a solution, powder or gel; (c) the composition comprises a mesh or network of fibers of the peptide; (d) the composition is applied to one or more surfaces of the substrate by spraying; (e) the one or more aquatic organisms is selected from corals, clams, sponges, or algae and/or (f) the substrate is selected from ceramics, 3D printed structures, marine structures, limestone, and limestone and/or ceramic composites.

32. (canceled)

33. (canceled)

34. (canceled)

35. The composition of claim 3331, wherein the peptide is self-assembled and/or the composition supports coral growth and restoration.

36. (canceled)

37. (canceled)

38. (canceled)

39. (canceled)

40. A method for promoting growth of coral comprising:
(i) providing a substrate for coral attachment;
(ii) applying a composition comprising an effective amount of one or more self-assembling peptides to the surface(s) of the substrate;
depositing coral upon the coated surface(s); and placing the substrate in an environment suitable for the growth of the coral.

41. The method of claim 40, wherein the one or more self-assembling peptides independently comprise one or more L-3,4-dihydroxyphenylalanine (L-dopa) residues and optionally, wherein the substrate is a 3D printed substrate.

42. (canceled)
